(19) United States
(12) Patent Application Publication
(10) Pub. No.: US 2011/0200650 A1
(43) Pub. Date: Aug. 18, 2011

(54) Non-Aerosol Personal Care Compositions Comprising An Anti-Irritation Agent

(76) Inventor: James Robert Schwartz, West Chester, OH (US)

(21) Appl. No.: 13/028,901

(22) Filed: Feb. 16, 2011

Related U.S. Application Data
(60) Provisional application No. 61/305,168, filed on Feb. 17, 2010.

(51) Int. Cl. A61K 8/18; A61K 8/58; A61K 8/27; A61K 8/72; A61K 8/02; A61K 8/81; A61K 8/73; A61K 8/46; A61Q 9/02

(52) U.S. Cl. 424/401; 424/73; 424/70.11; 424/70.15; 424/70.17; 424/70.16; 424/70.13; 424/70.21; 424/70.31

(57) ABSTRACT

A personal care composition suitable for use as a non-aerosol shave composition comprising an anti-irritation agent; a lathering surfactant; a carrier comprising water; and optional adjunct ingredients such as lubricants.

### CROSS REFERENCE TO RELATED APPLICATION

This application claims the benefit of U.S. Provisional Application No. 61/305,168, filed Feb. 17, 2010.

### FIELD OF THE INVENTION

The present invention relates to a personal care composition, preferably one in a non-aerosol form that contains an anti-irritation agent. The personal care composition is preferably a shave or hair removal preparation.

### BACKGROUND OF THE INVENTION

Over the past century, the forms of shave aids have changed and evolved. Shaving soaps and brushes were used in the early part of the 20^{th} century, where the soap was placed into a mug and rubbed with the brush to generate lather. After World War II, aerosol technology, developed to deliver pesticides, was reapplied and the first aerosol shave foams were born. These shave foams are composed of thin soap solutions and a volatile hydrocarbon that when mixed together in situ form a voluminous, creamy lather. Another popular form used today is the post foaming shave gel, developed in the late 70's. Amine-based soaps are combined with volatile hydrocarbons to form a clear, stable emulsion when kept under pressure. Once dispensed and mechanically agitated, these gels transform into thick foams. Although aerosols are by far the most widely used form of shave aid, they are almost exclusively based on soap technology. Soaps can be harsh to the skin, especially in hard water, and limit the types of ingredients that can be included due to the high pH of the product. Aerosol shave gels also require a package with an inner compartment, into which the shave gel is filled, and an outer chamber, which is filled with a high pressure gas that causes the shave gel to be dispensed when actuated.

Non-aerosol shave aids can be formulated over a wide pH range to allow for inclusion of skin benefit agents and can be packaged into much less expensive containers. The two main types of non-aerosol shave aids sold today are either emulsions (creams/lotions) or gels, which most commonly consist of polymer thickened surfactant systems. Even as many of these systems provide lubrication and/or a smooth shave, skin irritation remains a problem. A number of approaches to reduce irritation have been attempted, but have not resulted in satisfactory results. Moreover, as skin irritation indicates damage to the skin, the skin is left more susceptible to microbial infection. Thus, there is a need to treat the irritation of the skin as well as treat the skin for antimicrobials.

### SUMMARY OF THE INVENTION

One aspect of the present invention provides for a personal care composition suitable for use as a non-aerosol shave composition comprising: from about 0.01% to 5% of an anti-irritation agent; from about 5% to about 30% of a lathering surfactant; from about 50% to about 90% of a carrier comprising water; and optional adjunct ingredients such as lubricants.

### DETAILED DESCRIPTION OF THE INVENTION

In the embodiments disclosed herein, percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The present invention relates to a personal care composition, preferably one in a non-aerosol form that contains an anti-irritation agent. The personal care composition can be a shave or hair removal preparation. For example, in one embodiment, the personal care composition disclosed herein can be suitable for use as a non-aerosol shave composition. According to an example embodiment, the personal care composition can include from about 0.01% to 5% of an anti-irritation agent; from about 5% to about 30% of a lathering surfactant; from about 50% to about 90% of a carrier comprising water; and optional adjunct ingredients such as lubricants.

Without wishing to be bound by theory, it is believed that the compositions of the present invention surprisingly provide an antimicrobial benefit and reduce irritation. It is believed that zinc pyrithione, when added to these compositions, provides the synergistic benefit of reduced irritation, while providing an antimicrobial benefit. It is this unique combination of benefits provided by these compositions that result in an improved shaving experience.

### 1. Anti-Irritation Agent

In the personal care composition of the present invention, the anti-irritation agent is pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

The composition of the present invention optionally includes an effective amount of a zinc salt.

Preferred embodiments of the present invention include an effective amount of a zinc salt having an aqueous solubility within the composition of less than about 25%, by weight, at 25° C., more preferably less than about 20%; more preferably less than about 15%.

Preferred embodiments of the present invention include from 0.001% to 10% of a zinc salt; more preferably from 0.01% to 5%; more preferably still from 0.1% to 3%.

In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

### 2. Lathering Surfactants

The personal care composition, which can be a wash or cleansing composition (such as a shave prep composition), can comprise one or more lathering surfactants and a carrier such at water, at a total level of from about 60% to about 99.99%. A lathering surfactant defined herein to as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still being able to produce a lather.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants.

Concentrations of these surfactant are from about 10% to about 20%, alternatively from about 5% to about 25%, and alternatively from about 2% to about 30% by weight of the composition. To avoid skin irritation issues, the compositions containing anionic surfactants should have a ratio by weight of the composition of anionic surfactant to amphoteric and/or zwitterionic surfactant is from about 1.1:1 to about 1:1.5, alternatively from about 1.25:1 to about 1:2, and alternatively from about 1.5:1 to about 1:3.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine). Also useful are taurates which are based on taurine, which is also known as 2-aminoethanesulfonic acid, and to glutamates, especially those having carbon chains between C₈ and C₁₆.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants is from about 1% to about 10%, alternatively from about 0.5% to about 20% by weight of the composition. Non-limiting examples of suitable zwitterionic or amphoteric surfactants are described in U.S. Pat. Nos. 5,104,646 and 5,106,609.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Preferred lathering surfactants for use herein are the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

One suitable lathering surfactant is a polyglyceryl fatty ester. In one embodiment the polyglyceryl fatty ester surfactant has the formula:

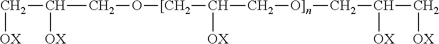

wherein n is 1 to 10, and X is a hydrogen atom or a long chain acyl group derived from a C₁₂₋₂₂ fatty acid or an N-fatty acyl-neutral amino acid, provided that at least one X is a long chain acyl group and no more than three X's are long chain acyl groups. In one embodiment, the polyglyceryl fatty ester surfactant is selected from the group consisting of: polyglyceryl-10 oleate, polyglyceryl-6 stearate, polyglyceryl-10 stearate, polyglyceryl-8 dipalmitate, polyglyceryl-10 dipalmitate, polyglyceryl-10 behenate, and polyglyceryl-12 trilaurate.

### 3. Carrier

The personal care compositions of the present invention also comprise a carrier. In one embodiment the carrier comprises water. The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the personal care composition comprises from about 50% to about 99.99%, preferably from about 60% to about 99.9%, more preferably from about 70% to about 98%, and even more preferably from about 80% to about 95% of the carrier by weight of the composition.

### 4. Adjunct Ingredients

a. Lubricants

In one embodiment, said personal care composition comprises at least one lubricant selected from: a lubricious water soluble polymer; a water insoluble particle, a hydrogel forming polymer, and a mixture thereof.

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Examples of suitable polyethylene oxides include PEG-23M, PEG-45M, and PEG-90M. The lubricious water soluble polymer can be at a level of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. Generally, the hydrogel-forming polymer will be included in the shaving composition in an amount of about 0.0005% to about 3%, or about 0.001% to about 0.5%, or about 0.002% to about 0.1%, by weight.

Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer. Glyceryl acrylate/acrylic acid copolymer is highly hydrophilic, has a molecular weight greater than 1 million daltons and generally includes a polyacrylic acid backbone partially esterified (typically about 50% esterified) with glycerin. It is believed that the glyceryl acrylate/acrylic acid copolymer forms a clathrate that holds water, which, upon release, supplies lubrication and moisturization to the skin. It has been found that shave gel compositions that include the glyceryl acrylate/acrylic acid copolymer have improved gel structure and reduced coefficient of friction (i.e., increased lubricity). See e.g. U.S. 2006/00257349 at ¶ 10.

The term “water dispersible”, as used herein, means that a substance is either substantially dispersible or soluble in water. The water dispersible surface active agent is preferably one that is capable of forming a lather, such as one or more of the optional lathering surfactants described in section 5 below (including but not limited to a soap, an interrupted soap, a detergent, an anionic surfactant, a non-ionic surfactant or a mixture of one or more of these.)

b. Polar Solvents

In one embodiment, the carrier comprises a polar solvent. The level of polar solvent can be from about 1% to about 20%, or from about 5% to about 10%. Polar solvents useful herein include polyhydric alcohols such as, 3-butylene glycol, propane diol, ethylene glycol, diethylene glycol, sorbitol, and other sugars which are in liquid form at ambient temperature glycerin, sorbitol, propylene glycol, butylene glycol, pentylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Polyols such as those containing from 2 to about 6 carbon atoms and from 2 to about 6 hydroxy groups are preferred (e.g., 1,3-propanediol, ethylene glycol, glycerin, and 1,2-propanediol) can also be used. The most preferred are Butylene, Pentylene or Hexylene Glycol and mixtures there of.

Without intending to be bound by theory, it is believed that the addition of one or more, polar solvents, allows for reduction in the viscosity and improvement in the clarity of the personal care composition while maintaining good lubrication.

c. Salycylic Acid

The personal care composition of the present invention may comprise a salicylic acid compound, its esters, its salts, or combinations thereof. In the compositions of the present invention, the salicylic acid compound preferably comprises from about 0.1% to about 5%, preferably from about 0.2% to about 2%, and more preferably from about 0.5% to about 2%, by weight of the composition, of salicylic acid.

d. Other Adjunct Ingredients

The compositions of the present invention may contain a variety of other ingredients that are conventionally used in given product types provided that they do not unacceptably alter the benefits of the invention. These ingredients should be included in a safe and effective amount for a personal care composition for application to skin.

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and to medicaments); and US 2005/0019356 (i.e. desquamation actives, anti-acne actives, chelators, flavonoids, and antimicrobial and antifungal actives). Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The personal care composition of the present invention is a non-aerosol composition. In one embodiment, the personal care composition is free or substantially free of a volatile post-foaming agent.

i. Conditioning Agents

The compositions of the present invention may comprise a conditioning agent selected from the group consisting of humectants, moisturizers, or skin conditioners, each can be present at a level of from about 0.01% to about 40%, more preferably from about 0.1% to about 30%, and even more preferably from about 0.5% to about 15% by weight of the composition. These materials include, but are not limited to, guanidine; urea; glycolic acid and glycolate salts (e.g. ammonium and quaternary alkyl ammonium); lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; sucrose polyester; petrolatum; and mixtures thereof.

Suitable moisturizers, also referred to in the present invention as humectants, include urea, guanidine, glycolic acid and glycolate salts (e.g. ammonium and quaternary alkyl ammonium), lactic acid and lactate salts (e.g. ammonium and quaternary alkyl ammonium), aloe vera in any of its variety of forms (e.g. aloe vera gel), polyhydroxy alcohols (such as sorbitol, glycerol, hexanetriol, propylene glycol, hexylene glycol and the like), polyethylene glycol, sugars and starches, sugar and starch derivatives (e.g. alkoxylated glucose), hyaluronic acid, lactamide monoethanolamine, acetamide monoethanolamine, and mixtures thereof.

ii. Thickening Agents (Including Thickeners and Gelling Agents)

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting to classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); crosslinked polyacrylate polymers (including both cationic and nonionic polymers, such as described in U.S. Pat. Nos. 5,100,660; 4,849,484; 4,835,206; 4,628,078; 4,599,379, and EP 228,868); polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); polyacrylamide polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); polysaccharides (nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof); gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. No. 6,967,027 to Heux et al.; U.S. Pat. No. 5,207,826 to Westland et al.; U.S. Pat. No. 4,487,634 to Turbak et al.; U.S. Pat. No. 4,373,702 to Turbak et al. and U.S. Pat. No. 4,863,565 to Johnson et al., U.S. Patent Publ. No. 2007/0027108 to Yang et al.)

### 5. Compositional pH

The personal care composition of the present invention preferably has a pH of less than about 9, more preferably less than about 7. In one embodiment the composition has a pH of less than about 5, or less than about 4. In one preferred embodiment the composition has a pH range of from about 2.5 to about 4.5 Suitable lathering surfactants for use at pH levels below about 4 can be selected from the group consisting of alkyl sulfonates, pareth sulfonates, sulfobetaines, alkylhydroxysultaines, alkylglucosides and mixtures thereof.

### 6. Methods of Use

The present composition is preferably a hair removal preparation such as a non-aerosol shave foam or gel. The personal care compostion of the present invention can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

The present invention includes a method of treating skin irritation which can be the result of one or more of said hair removal technologies, said method comprising: applying a personal care composition to a portion of skin to form a portion of treated skin, said personal care composition comprising a naphthalenyl ketone; and down regulating at least one pain receptor in proximity with said portion of treated skin. In one embodiment, said method further comprises a step of at least partially removing hair from said portion of skin prior to the step of applying the personal care composition. In another embodiment, said method further comprises a step of at least partially removing hair from said portion of treated skin after to the step of applying the personal care composition.

### 7. Methods of Making

One embodiment of the present invention provides for a method of making the personal care composition. One method for making the present composition comprises:

a. Weighing out the water in a vessel sufficient to hold the entire batch
b. Inserting an overhead mixer with impeller into the vessel and increase agitation to create a vortex
c. Pre-blending the cationic polysaccharide and PEG polymer powders
d. Adding the polymer blend into the vortex until incorporated (addition can be done by sprinkling)
e. Begin heating batch to 70 C to hydrate the polymers, increasing rpms to maintain good mixing
f. Once the batch is at 70 C, adding the surfactant(s) and mix until uniform
g. Begin cooling batch to below 45 C
h. Once below 45 C, adding the perfume, preservatives, glycerin/glycols and other temperature-sensitive additives
i. Cooling to below 35 C and QS with water
For product with acne control actives, add the salicylic acid during STEP f. addition.
For product with water-soluble actives, add to the batch during STEP h. additions
For product with oil-soluble actives, add to the batch during STEP f. additions

### 8. Composition Characteristics

a. Turbidity

In one embodiment, the personal care composition comprises a turbidity of below about 320 NTU, alternatively less than about 250 NTU, alternatively less than about 200 NTU, alternatively less than about 150 NTU, alternatively less than about 100 NTU, as measured by Turbimeter test method disclosed herein. Compositions with a turbidity below about 150, alternatively below about 100 are considered “clear” while those with a turbidity below about 320, alternatively below about 250 are “translucent.”

As used herein, turbidity is determined using a Hach Model 2100AN Turbidimeter (“Turbimeter”), by Hach Company, Loveland, Colo. StablCal is a trademark of Hach Company.

Turbidimeter Turbidity Method: The Turbidimeter measures the turbidity from 0.1 NTU to 7500 NTU. The Turbidimeter operates on the nephelometric principle of turbidity measurement. The Turbidimeter's optical system includes a tungsten-filament lamp, a 90° detector to monitor scattered light and a transmitted light detector. The Turbidimeter's microprocessor calculates the ratio of the signals from the 90° and of transmitted light detectors. This ratio technique corrects for the interferences from color and or light absorbing materials and compensates for fluctuations in the lamp intensity.

Calibration is by StablCal® Secondary standards provided with the Turbidimeter. The undiluted sample is contained in the sample cell, the outer cell wall is wiped free of water and finger prints. A thin coat of silicone oil is applied to the outer wall of the sample cell in order to mask minor imperfections and scratches on the sample cell wall, which may contribute to turbidity or stray light. A measurement is taken and result is displayed in NTU units. All samples are equilibrated and measured at 25° C. The samples are measured within 24 h after making b. Viscosity

Viscosity measurements are determined using a Brookfield viscometer with Helipath using a spindle T-C @ 5 rpms. Sample product is poured into a glass jar having dimensions of about 11 cm high by 5 cm wide and is filled to a depth of at least 8 cm and allowed to set for 24 hours prior to measurement. The T-C spindle was attached to the viscometer and placed over the top of the sample. The Helipath is activated and the spindle was slowly lowered into the product while rotating at 5 rpms. The viscosity is monitored as the spindle continued further into the product to check for uniformity of the sample. The final viscosity reading is taken as the spindle was approximately halfway through the sample.rpms.

c. In Shave Lubrication Test

It has been found that the personal care composition of the present invention provides for an in shave lubrication benefit as shown by reduced friction as measured by the In Shave Lubrication “ISL” Test defined herein. Reducing friction is important during the shave because a high friction skin surface results in bulging of the skin. When the skin bulges, the blade is more likely to engage the skin, increasing the chance for skin irritation. Therefore, by reducing friction the product helps protect the skin. In addition, lower friction results in less drag on the skin, which can also be a potential source of irritation. This method enables measurement of the coefficient of friction (CoF) of a shave preparation.

In Shave Lubrication Test Method: An apparatus designed to simulate lubrication during the shaving process is connected to an instrument capable of measuring frictional forces (for example, an Instron-type instrument) and containing a load cell of about 1 kg to about 100 kg. The rinsing apparatus comprises: 1) an air-activated clamping device capable of opening and closing to deliver pressures of about 10 psi to about 70 psi to simulate the pressure exerted by hands on hair during rinsing 2) keratinous tissue models as described herein affixed to two opposing sides of the clamping device and 3) one or more spray nozzles capable of delivering water flow rates of from about 50 ml/min. to about 1000 mL/min., for simulating shower conditions.

Procedure: Attach the rinsing apparatus to the base of a Stable Micro Systems TA XT Plus™ Texture Analyzer (TA) equipped with a 30 kg load cell, centering or aligning the clamps perpendicular to the load cell. Adjust water flow rate to approximately 200 ml/min and the water to temperature to 103° F.+/−2° F. Set the air pressure for the TA clamps to approximately 30 psi. Set the instrument measurement settings as follows: TA settings, tension compression, test speed-10.0 mm/sec for 130 mm long pull. Set the macro for a total of 10 strokes. Run the first five strokes without the water on, then manually turn on the water for 2 min 15 sec for the second five strokes. During the test, data (g of force) will only be collected during the upward pull of the treated KTM, not on the return. Cover the pads on both the front and back side of the piston with a polyurethane skin pad (see JP2006233367 for details).

Wet a 2 inch by 9 inch piece of nonwoven KTM under hot (−103° F.+/−2° F.) tap water for 30 seconds. Place 2 g+/−0.1 g of aerosol shave gel or 1 g+/−0.1 g of aerosol shave foam onto the nonwoven KTM and gently lather and spread by hand for 30 seconds. Rub excess foam on the back of the nonwoven KTM. Load the nonwoven KTM into the TA and start the test macro. At the end of the fifth stroke, turn on the rinse water. Initiate a test sequence which 1) instructs the instrument to raise the load cell to which the KTM is attached, at a rate of about 10 mm/sec 2) opens the clamps, and 3) instructs the instrument to lower the load cell. Repeat this sequence until a predetermined number of sequences may be executed, for example, 10. Between each sample, wipe the polyurethane skin pads with a piece of nonwoven KTM and an alcohol wipe to remove any possible build-up from the previous test. By calculating the total friction in grams of force (or other suitable unit of force) for dry friction and rinse friction, products may be ranked relative to each other to assess which products would be expected to have the most pleasant feel.

“KTM” as defined herein means a “Keratinous tissue mimic” which refers to one or more artificial substrates which may have one or more physical properties representative of keratinous tissue. The KTM used for the purposes of this application is TENCEL from Lenzing, Inc. Additional details on other KTMs is disclosed in Section I of U.S. Ser. No. 61/239,908 to Battaglia et al, filed Sep. 4, 2009.

All percentages disclosed herein, unless otherwise stated, are by weight of the named material itself that is found in the compositions, thereby excluding for example the weight associated with carriers, impurities and by-products found in the raw material.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations to were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a”, “an” and “the” mean “one or more”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a. from about 0.01% to 5% of an anti-irritation agent;
b. from about 5% to about 30% of a lathering surfactant;
c. from about 50% to about 90% of a carrier comprising water; and
d. optional adjunct ingredients.

2. The personal care composition of claim 1, wherein said personal care composition comprises from about 0.25% to about 2.5% of an anti-irritation agent.

3. The personal care composition of claim 1, wherein said anti-irritation agent comprises zinc pyrithione.

4. The personal care composition of claim 1, wherein said personal care composition further comprises from 0.001% to 10% of a zinc salt.

5. The personal care composition of claim 4, wherein said zinc salt comprises zinc oxide.

6. The personal care composition of claim 1, further comprising a lubricant selected from: a lubricious water soluble polymer; a water insoluble particle, a hydrogel forming polymer, and a mixture thereof.

7. The personal care composition of claim 6, wherein said lubricious water soluble polymer comprises at least one of a polyethylene oxide, a polyvinylpyrrolidone, a polyacrylamide, and a mixture thereof.

8. The personal care composition of claim 6, wherein said water insoluble particle comprises at least one of an inorganic particle, an organic polymer particle, and a mixture thereof.

9. The personal care composition of claim 6, wherein said hydrogel-forming polymer comprises at least one of: a polyacrylic acid or polymethacrylic acid partially esterified with a polyhydric alcohol; a hydrophilic polyurethane; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

10. The personal care composition of claim 1, further comprising from about 1% to about 20% of at least one polar solvent.

11. The personal care composition of claim 10, wherein said polar solvent is selected from the group consisting of: glycerin, 1,3-butylene glycol, propylene glycol, hexylene glycol, propane diol, ethylene glycol, diethylene glycol, dipropylene glycol, diglycerin, sorbitol, and a mixture thereof.

12. The personal care composition of claim 1, wherein said lather surfactant comprises from about 5% to about 20% of alkyl sulfonates, pareth sulfonates, sulfobetaines, alkylhydroxysultaines, alkyl glucosides, alkyl polyglucosides and mixtures thereof.

13. The personal care composition of claim 1, further comprising from about 0.5 to about 2.0% of a salicylic acid.

14. The personal care composition of claim 13, having a compositional pH of from about 2.5 to about 4.5.
